(19) United States
(12) Patent Application Publication
(10) Pub. No.: US 2025/0230466 A1
(43) Pub. Date: Jul. 17, 2025

(54) TISSUE-SPECIFIC PROMOTER AND USE THEREOF

(71) Applicant: BEIJING MEIKANG GENO-IMMUNE BIOTECHNOLOGY CO., LTD., Beijing (CN)

(72) Inventors: Lung-Ji Chang, Beijing (CN); Jie Gong, Beijing (CN)

(21) Appl. No.: 18/705,431

(22) PCT Filed: Oct. 20, 2022
(86) PCT No.: PCT/CN2022/126404

§ 371 Date: Apr. 26, 2024

(30) Foreign Application Priority Data
Oct. 28, 2021 (CN) 202111261085.5

(51) Int. Cl. C12N 15/86; A61K 48/00; A61P 7/04; C07K 14/755

(52) U.S. Cl. CPC C12N 15/86; A61K 48/0058; A61P 7/04; C07K 14/755; C12N 2740/15043; C12N 2830/008

(57) ABSTRACT

Provided are a tissue-specific promoter and use thereof. The tissue-specific promoter has a nucleic acid sequence comprising more than 80% of the sequence as shown in SEQ ID NO: 1, SEQ ID NO: 2, SEQ ID NO: 3 or SEQ ID NO: 4. The tissue-specific promoter can promote the specific expression of a coding gene in endothelial cells (ECs) or megakaryocyte-platelet cells and can be applied to gene therapy in which a gene is required to be specifically expressed in ECs or megakaryocyte-platelet cells, ensuring a therapeutic effect, reducing a risk of immune rejection and saving a therapeutic cost.

### TECHNICAL FIELD

The present application belongs to the field of biotechnology and relates to a tissue-specific promoter and use thereof.

### BACKGROUND

Gene therapy refers to the introduction of an exogenous gene into target cells in order to correct or compensate for a defective gene or an abnormal gene, for the purpose of treating a disease caused by the defective gene or the abnormal gene. However, non-specific expression often occurs, that is, the exogenous gene is widely expressed in human body and causes immune rejection in the human body, which is one of the problems difficult to resolve in the clinics.

For example, hemophilia A (HA), also known as hereditary antihemophilic globulin deficiency or FVIII (F8) deficiency, is a coagulation disorder caused by a genetic defect in the coagulation factor VIII gene (a FVIII gene or a F8 gene). The current treatment of HA mainly includes methods such as protein replacement therapy through plasma-derived coagulation factors or exogenously-produced recombinant proteins, and gene therapy. An analysis of the human F8 gene revealed an obvious domain structure for the protein, represented as A1-A2-B-A3-C1-C2. The B domain is encoded by large exons with a highly conserved region consisting of asparagine (N)-linked oligosaccharides. Miao et al. showed that a partial B domain deletion, leaving an N-terminal 226-amino acid stretch containing six intact asparagine-linked glycosylation sites, was able to increase in vitro F8 secretion by 10-fold (see Miao, H. Z., Sirachainan, N., Palmer, L., Kucab, P., Cunningham, M. A. et al. Bioengineering of coagulation factor VIII for improved secretion. Blood, 2004, 103 (9), 3412-3419.). At present, however, the gene therapy method using the B-domain-deleted F8 gene (F8-BDD) has the problems such as low protein secretion and function, low transduction efficiency of F8 viral vectors and antibody formation associated with inhibitory reaction (immune rejection).

Therefore, how to achieve a tissue-specific in vivo expression of a therapeutic gene to avoid the immune rejection is an urgent problem to be solved in the field of gene therapy.

### SUMMARY

In view of deficiencies and actual requirements of the existing art, the present application provides a tissue-specific promoter and use thereof. The tissue-specific promoter can promote a specific expression of a coding gene in endothelial cells (ECs) megakaryocytes or platelets, and reduce an ectopic expression in unrelated tissue cells. The tissue-specific promoter can be applied to gene therapy in which a gene is required to be specifically expressed in ECs, effectively reducing antibody response and inhibitor reaction.

To achieve the above object, the present application uses the technical solutions below.

In a first aspect, the present application provides a tissue-specific promoter, which has a nucleic acid sequence comprising more than 80% of the sequence as shown in SEQ ID NO: 1, SEQ ID NO: 2, SEQ ID NO: 3 or SEQ ID NO: 4.

SEQ ID NO: 1:
gcggccgctccaaaaatatccttccatcacactccccatcttgtgctctgatttactaaacggccctgggccctctctttctcagggtctctgc
ttgcccagctatataataaaacaagtttgggacttcccaaccattcacccatggaaaaacagaagcaactcttcaaaggacagattcccaggat
ctgccctgggagattccaaatcagttgatctggggtgagcccagtcctctgtagtttttagaagctcctcctatgtctctcctggtcagcagaa
tcttggcccctcccttccccccagcctcttggttcttctgggctctgatccagcctcagcgtcactgtcttccacgcccctctttgattctcgt
ttatgtcaaaagccttgtgaggatgaggctgtgattatccccattttacagatgaggaaactgtggctccaggatgacacaactggccagaggt
cacatcagaagcagagctgggtcacttgactccacccaatatccctaaatgcaaacatcccctacagaccgaggctggcaccttagagctggag
tccatgcccgctctgaccaggagaagccaacctggtcctccagagccaagagcttctgtccctttcccatctcctgaagcctccctgtcacctt
taaagtccattcccacaaagacatcatgggatcaccacagaaaatcaagctctggggctaggctgaccccagctagatttttggctcttttata
ccccagctgggtggacaagcaccttaaacccgctgagcctcagcttcccgggctataaaatgggggtgatgacacctgcctgtagcattccaag
gagggttaaatgtgatgctgcagccaagggtccccacagccaggctctttgcaggtgctgggttcagagtcccagagctgaggccgggagtagg
ggttcaagtggggtgccccaggcagggtccagtgccagccctctgtggagacagccatccggggccgaggcagccgcccaccgcagggcctgcc
tatctgcagccagcccagccctcacaaaggaacaataacaggaaaccatcccagggggaagtgggccagggccagctggaaaacctgaagggga
ggcagccaggcctccctcgccagcggggtgtggctcccctccaaagacggtcggctgacaggctccacagagctccactcccggggatcc.
SEQ ID NO: 2:
gcggccgcctccttcccctgggcctaaggatatcttggctggaagctctgctctgaaaaggggcatggccaaactttcactagggctcttc
gttggggagcacgatggacaaaagccttcttggggctaggcaggtcacttcaaacttggagccgccaaatattttgggaaatagcgggaatgct
ggcgaactgggcaagtgcgttttctgattaagagcaaccagattcagctttttaaactacaattatactggccaaacaaaatacccttatacaa
aaaccaaaactactggcaggagtcgctgccagcttgcgacccggcatacttggctgagtatccgcttctcccttgtggctccaaactgctgcag
attctcggccacttcagacgcgcgcgatggcgaagagggtcctgcactttgacgcgcctggtgagggagcggtgctcttcgcagcgctcctggt
gatgctccccaaatttcggggaccggcaagcgattaaatcttggagttgctcagcgcccgttaccgagtactttttatttacaccagaaacaaa
gttgttgctctgggatgttctctcctgggcgacttggggcccagcgcagtccagttgtgtggggaaatggggagatgtaaatgggcttggggag
ctggagatccccgccgggtacccgggtgaggggcggggctggccgcacgggagagcccctcctccgccccggccccgccccgcatggccccgc
ctccgcgctctagagtttcggctccagctcccaccctgcactgagtcccgggaccccgggagagcggtcagtgtgtggtcgctgcgtttcctct
gcctgcgccgggcatcacttgcgcgccgcagaaagtccgtctggcagcctggatatcctctcctaccggcacccgcagacgcccctgcagccg
ccggtcggcgcccgggctccctagccctgtgcgctcaactgtcctgcgctgcggggtgccgcgagttccacctccgcgcctccttctctagaca
ggcgctgggagaaagaaccggctcccgagttctgggcatttcgcccggctcgaggtgccccggggatcc.
SEQ ID NO: 3:
gcggccgctgtgaacggaccaagagtaaacagtgtgctcaatgctgtgcctacgtgtgttagcccacgcggccagcctgaggagtcag
ggaaggctcccctaggcaaagcccccaaccagaatcaagtcttaatggttaaagagctccatcacccaaaaaggattgagggcctaccttcaac
tgaacagctaatgcataatctcagaaactgtgagtcaaaattccctggaataactccactttatccccaatctccttgccacctagaccaaggt
ccattcaccaccctgtccccagcactgactgcactgctgtggccacactaaagcttggctcaagacggaggaggagtgaggaagctgctgcacc
aatatggctggttgaggccgcccaaggtcctagaaggaggaagtgggtaaatgccatatccaaaaagatacagaagcctcaggttttatcgggg
gcagcagcttccttctccttccccgacctgtggccaagtcacaaagcaccacagctgtacagccagatgggggaagggaggagattagaactgt
aggctagagtagacaagtatggaccagttcacaatcacgctatcccaagcagaaagtgatggtggcttggactagcacggtggtagtagagatg
gggtaaagattcaagagacatcattgataggcagaaccaataggacatggtaataaactattctcaggaaaggggaggagtcatggctttcagc
catgagcatccaccctctgggtggcctcacccacttcctggcaattctagccaccatgagtccaggggctatagccctttgctctgcccgttgc
tcagcaagttacttggggttccagtttgataagaaaagacttcctgtggaggaatctgaagggaaggaggaggagctggcccattcctgcctgg
gaggttgtggaagaaggacccggggatcc.
SEQ ID NO: 4:
gcggccgctctgggattacaggcatgagccacgcgcccggccctggagaggtttttaaaagatggcagaaggctgtttggaggagtcc
acccccatctcccctgtgtaaaaggaaagcggaagagagaaccacaaagagggcctgggggaaagccgtggagtgaggcgataagggctt
gtgtccaggggattcccggtcactggaatccctatcaggcctgcatttcctcctcacccccatccccttccttgccactggcttagtcctccat
ggggctagaagagagaaggacggagtcgagtggcaccctagaagacgctctgtgccttcggaggatcc.

The present application has creatively designed tissue-specific promoters, including promoters VEC (SEQ ID NO: 1 or having at least 80% homology with SEQ ID NO: 1) and KDR (SEQ ID NO: 2 or having at least 80% homology with SEQ ID NO: 2) that promote a specific expression of a gene in ECs, and promoters ITGA (SEQ ID NO: 3 or having at least 80% homology with SEQ ID NO: 3) and Gp (SEQ ID NO: 4 or having at least 80% homology with SEQ ID NO: 4) that promote a specific expression of a gene in megakaryocyte-platelet cells. A coding gene can be specifically expressed in ECs or megakaryocyte-platelet cells by employing the tissue-specific promoter of the present application. Therefore, the tissue-specific promoter of the present application can be effectively applied to gene therapy in which a target coding gene is required to be specifically expressed in ECs or megakaryocyte-platelet cells, reducing the risk of immune rejection and saving a therapeutic cost, such as gene therapy for HA.

Preferably, the tissue-specific promoter has a nucleic acid sequence as shown in SEQ ID NO: 1, SEQ ID NO: 2, SEQ ID NO: 3 or SEQ ID NO: 4.

In a second aspect, the present application provides a gene expression cassette. The gene expression cassette includes the tissue-specific promoter according to the first aspect and a coding gene.

Preferably, the coding gene includes a coding gene of a recombinant coagulation factor VIII.

Preferably, the coding gene of the recombinant coagulation factor VIII comprises a nucleic acid sequence as shown in SEQ ID NO: 5.

SEQ ID NO: 5:
atgcagatcgaactgagcacctgcttcttcctgtgtctcctgagattctgctttagtgctaccagacggtattacctgggagccgtcgagctg
agttgggattacatgcagtccgacctcggagaactgcctgtggatgcacgctttccaccaagagtgcctaagtcattcccattcaacacctcag
tcgtgtataagaagactctgttcgtcgagtttactgatcacctgttcaatatcgctaaacctagaccaccctggatgggactgctgggtcctac
aatccaggcagaggtctatgacactgtggtgattacacttaagaacatggcttcccatcctgtcagtcttcatgctgttggtgtatcctactgg
aaagcttctgagggagctgaatatgatgatcagaccagtcaaagggagaaagaagatgataaagtcttccctggtggaagccatacatatgtct
ggcaggtcctgaaagagaatggtccaatggcctctgacccactgtgccttacctactcatatctttctcatgtggacctggtaaaagacttgaa
ttcaggcctcattggagccctactagtatgtagagaagggagtctggccaaggaaaagacacagaccttgcacaaatttatactactttttgct
gtatttgatgaagggaaaagttggcactcagaaacaaagaactccttgatgcaggatagggatgctgcatctgctcgggcctggcctaaaatgc
acacagtcaatggttatgtaaacaggtctctgccaggtctgattggatgccacaggaaatcagtctattggcatgtgattggaatgggcaccac
tcctgaagtgcactcaatattcctcgaaggtcacacatttcttgtgaggaaccatcgccaggcgtccttggaaatctcgccaataactttcctt
actgctcaaacactcttgatggaccttggacagtttctactgttttgtcatatctcttcccaccaacatgatggcatggaagcttatgtcaaag
tagacagctgtccagaggaaccccaactacgaatgaaaaataatgaagaagcggaagactatgatgatgatcttactgattctgaaatggatgt
ggtcaggtttgatgatgacaactctccttcctttatccaaattcgctcagttgccaagaagcatcctaaaacttgggtacattacattgctgct
gaagaggaggactgggactatgctcccttagtcctcgcccccgatgacagaagttataaaagtcaatatttgaacaatggccctcagcggattg
gtaggaagtacaaaaaagtccgatttatggcatacacagatgaaacctttaagactcgtgaagctattcagcatgaatcaggaatcttgggacc
tttactttatggggaagttggagacacactgttgattatatttaagaatcaagcaagcagaccatataacatctaccctcacggaatcactgat
gtccgtcctttgtattcaaggagattaccaaaaggtgtaaaacatttgaaggattttccaattctgccaggagaaatattcaaatataaatgga
cagtgactgtagaagatgggccaactaaatcagatcctcggtgcctgacccgctattactctagtttcgttaatatggagagagatctagcttc
aggactcattggccctctcctcatctgctacaaagaatctgtagatcaaagaggaaaccagataatgtcagacaagaggaatgtcatcctgttt
tctgtatttgatgagaaccgaagctggtacctcacagagaatatacaacgctttctccccaatccagctggagtgcagcttgaggatccagagt
tccaagcctccaacatcatgcacagcatcaatggctatgtttttgatagtttgcagttgtcagtttgtttgcatgaggtggcatactggtacat
tctaagcattggagcacagactgacttcctttctgtcttcttctctggatataccttcaaacacaaaatggtctatgaagacacactcacccta
ttcccattctcaggagaaactgtcttcatgtcgatggaaaacccaggtctatggattctggggtgccacaactcagactttcggaacagaggca
tgaccgccttactgaaggtttctagttgtgacaagaacactggtgattattacgaggacagttatgaagatatttcagcatacttgctgagtaa
aaacaatgccattgaaccaagaagcttttctcagaatcctcctgtcctcaaacgccatcaacgggagattacacggaccacactccaaagcgat
caggaggagatcgactatgacgataccatatctgtggaaatgaagaaagaggacttcgacatctacgacgaagatgagaaccaaagtccaagat
ccttccagaagaagactaggcactacttcatcgctgccgtggaacgcctctgggattacggaatgtccagttctccacatgtcctcaggaatag
ggcacagtctggctctgttccacagtttaagaaagttgtctttcaggagttcacagatggctcattcactcaaccactgtatagaggcgaactg
aatgagcacctgggactgctgggtccctacatcagagccgaagtggaggataacattatggtcacctttcggaaccaagcctccaggccataca
gtttctacagttctctgatctcatacgaggaagatcagaggcaaggagcagaaccaaggaagaacttcgtgaaaccaaacgagacaaagaccta
tttctggaaagttcagcatcatatggcacccactaaagatgagtttgactgcaaagcctgggcttatttctctgatgttgacctggaaaaagat
gtgcactcaggcctgattggaccccttctggtctgccacactaacacactgaaccctgctcatgggagacaagtgacagtacaggaatttgctc
tgtttttcaccatctttgatgagaccaaaagctggtacttcactgaaaatatggaaagaaactgcagggctccctgcaatatccagatggaaga
tcccacttttaaagagaattatcgcttccatgcaatcaatggctacataatggatacactacctggcttagtaatggctcaggatcaaaggatt
cgatggtatctgctcagcatgggcagcaatgaaaacatccattctattcatttcagtggacatgtgttcactgtacgaaaaaaagaggagtata
aaatggcactgtacaatctctatccaggtgtttttgagacagtggaaatgttaccatccaaagctggaatttggcgggtggaatgccttattgg
cgagcatctacatgctgggatgagcacactttttctggtgtacagcaataagtgtcagactcccctgggaatggcttctggacacattagagat
tttcagattacagcttcaggacaatatggacagtgggccccaaagctggccagacttcattattccggatcaatcaatgcctggagcaccaagg
agcccttttcttggatcaaggtggatctgttggcaccaatgattattcacggcatcaagacccagggtgcccgtcagaagttctccagcctcta
catctctcagtttatcatcatgtatagtcttgatgggaagaagtggcagacttatcgaggaaattccactggaaccttaatggtcttctttggc
aatgtggattcatctgggataaaacacaatatttttaaccctccaattattgctcgatacatccgtttgcacccaactcattatagcattcgca
gcactcttcgcatggagttgatgggctgtgatttaaatagttgcagcatgccattgggaatggagagtaaagcaatatcagatgcacagattac
tgcttcatcctactttaccaatatgtttgccacctggtctccttcaaaagctcgacttcacctccaagggaggagtaatgcctggagacctcag
gtgaataatccaaaagagtggctgcaagtggacttccagaagacaatgaaagtcacaggagtaactactcagggagtaaaatctctgcttacca
gcatgtatgtgaaggagttcctcatctccagcagtcaagatggccatcagtggactctcttttttcagaatggcaaagtaaaggtttttcaggg
aaatcaagactccttcacacctgtggtgaactctctagacccaccgttactgactcgctaccttcgaattcacccccagagttgggtgcaccag
attgccctgaggatggaggttctgggctgcgaggcacaggacctctactga.

In a specific example of the present application, the tissue-specific promoter of the present application is used for initiating the expression of the coding gene of the recombinant coagulation factor VIII. The FVIII gene is expressed in ECs (such as hepatic sinusoidal ECs) or megakaryocytes to reduce the ectopic expression of FVIII protein in vivo and reduce antibody response and inhibitor reaction, effectively performing gene therapy for HA.

In a third aspect, the present application provides a recombinant expression vector. The recombinant expression vector includes the tissue-specific promoter according to the first aspect.

Preferably, the recombinant expression vector includes a viral vector or a plasmid vector containing the tissue-specific promoter according to the first aspect.

Preferably, the viral vector includes a lentiviral vector pEGWI.

Preferably, the recombinant expression vector further includes a coding gene.

Preferably, the coding gene includes a coding gene of a recombinant coagulation factor VIII.

Preferably, the 5′ splice donor site of the lentiviral vector pEGWI is mutated.

Preferably, the enhancer in the U3 region of the lentiviral vector pEGWI is deleted.

Preferably, the U3 region of the lentiviral vector pEGWI contains an insulator.

In the present application, the lentiviral vector pEGWI is modified. The wild-type 5′ splice donor site is mutated, the enhancer in the U3 region is deleted, and an insulator (a cHS4 insulator) is added in the U3 region, which not only can effectively improve transduction efficiency and expression efficiency of pEGWI so that the use cost of the vector is reduced, but can also improve safety.

In a specific example of the present application, the tissue-specific promoter and the coding gene of the recombinant coagulation factor VIII are co-inserted into the lentiviral vector pEGWI. Then the lentiviral vector is introduced into a body through direct intravenous injection so that the gene of the coagulation factor VIII is efficiently delivered and expressed specifically, effectively ensuring a therapeutic effect, reducing a risk of immune rejection, saving a therapeutic cost and achieving high-efficiency HA gene therapy.

In a fourth aspect, the present application provides a recombinant lentivirus. The recombinant lentivirus contains the recombinant expression vector according to the third aspect.

In a fifth aspect, the present application provides a recombinant cell. The recombinant cell contains the tissue-specific promoter according to the first aspect.

Preferably, the gene expression cassette according to the second aspect is integrated into a genome of the recombinant cell.

Preferably, the recombinant cell contains the recombinant expression vector according to the third aspect.

In a sixth aspect, the present application provides a method for preparing the recombinant cell according to the fifth aspect. The method includes the steps below:

introducing the tissue-specific promoter according to the first aspect, the gene expression cassette according to the second aspect, the recombinant expression vector according to the third aspect or the recombinant lentivirus according to the fourth aspect into a host cell to obtain the recombinant cell.

Preferably, the introduction is carried out by a method which includes any one of electrical transduction, a viral vector system, a non-viral vector system or direct gene injection.

Preferably, the host cell includes a hematopoietic stem cell.

In a seventh aspect, the present application provides a pharmaceutical composition. The pharmaceutical composition includes any one or a combination of at least two of the tissue-specific promoter according to the first aspect, the gene expression cassette according to the second aspect, the recombinant expression vector according to the third aspect, the recombinant lentivirus according to the fourth aspect or the recombinant cell according to the fifth aspect.

Preferably, the pharmaceutical composition further includes any one or a combination of at least two of a pharmaceutically acceptable carrier, excipient or diluent.

In an eighth aspect, the present application provides use of the tissue-specific promoter according to the first aspect, the gene expression cassette according to the second aspect, the recombinant expression vector according to the third aspect, the recombinant lentivirus according to the fourth aspect, the recombinant cell according to the fifth aspect or the pharmaceutical composition according to the seventh aspect in preparation of a drug for a tissue-specific gene therapy.

Compared with the existing art, the present application has the following beneficial effects:
(1) The tissue-specific promoter of the present application can promote the specific expression of the coding gene in ECs or megakaryocyte-platelet cells and can be applied to the gene therapy in which the gene is required to be specifically expressed in ECs or megakaryocyte-platelet cells, ensuring the therapeutic effect, reducing the risk of immune rejection and saving the therapeutic cost.
(2) In the present application, the tissue-specific promoter, the coding gene of the coagulation factor VIII and the lentiviral vector are used for constructing an expression vector. The expression vector can be successfully expressed in HA mice, correct the hemorrhagic phenotype of the HA mice to a certain extent and has a low antibody response, which is of great significance for ensuring effectiveness of gene therapy and lays a foundation for achieving faster relief from a symptom of HA and more comprehensive and durable gene therapy.

### BRIEF DESCRIPTION OF DRAWINGS

FIG. 1 is a structure diagram of a lentiviral vector pEGWI.

FIG. 2 is a structure diagram of different tissue-specific promoters and F8-BDD genes in a lentiviral vector.

FIG. 3 is a diagram illustrating vector copy numbers (VCNs) of recombinant lentiviruses in ECs and megakaryocytes.

FIG. 4 is a diagram illustrating results of analyzing an amount of expressed fluorescence in cells transduced with a recombinant lentivirus LV-wasabi.

FIG. 5 is a diagram illustrating results of an amount of expressed protein in cells transduced with a recombinant lentivirus LV-F8-BDD.

FIG. 6 is a diagram illustrating detection results of an in vitro plasma substrate luminescence method.

FIG. 7 is a schematic diagram of a treatment process of HA mice.

FIG. 8 is a diagram illustrating detection results of a positive coagulation factor VIII in blood cells of mice.

FIG. 9 is a diagram illustrating activity of a coagulation factor VIII in mice.

FIG. 10 is a diagram illustrating detection results of an enzyme-linked immunosorbent assay (ELISA) in plasma of mice.

### DETAILED DESCRIPTION

To further elaborate on the technical means adopted and effects achieved in the present application, the present application is further described below in conjunction with examples and drawings. It is to be understood that the specific examples set forth below are intended to explain the present application and not to limit the present application.

Experiments without specific techniques or conditions noted in the examples are conducted according to techniques or conditions described in the literature in the art or a product specification. The reagents or instruments used herein without manufacturers specified are conventional products commercially available from proper channels.

HA is used as an example in the examples of the present application, and it is proved that a tissue-specific promoter of the present application can be effectively applied to gene therapy and effectively reduce antibody response and inhibitor reaction against the coagulation factor.

### Example 1

A lentiviral vector carrying a specific promoter of the present application and F8 gene was constructed. Specifically, the present example includes the steps below.

(1) A structure diagram of lentiviral vector pEGWI is shown in FIG. 1. The wild-type 5′ splice donor site was mutated, the enhancer in U3 was deleted, and an insulator (a cHS4 insulator) was added in U3. For a specific modification method, see “Contributions of Viral Splice Sites and cis-Regulatory Elements to Lentivirus Vector Function, Cui et al. Journal of Virology, July 1999, p. 6171-6176”.

(2) Insertion of different tissue-specific promoters and F8-BDD gene

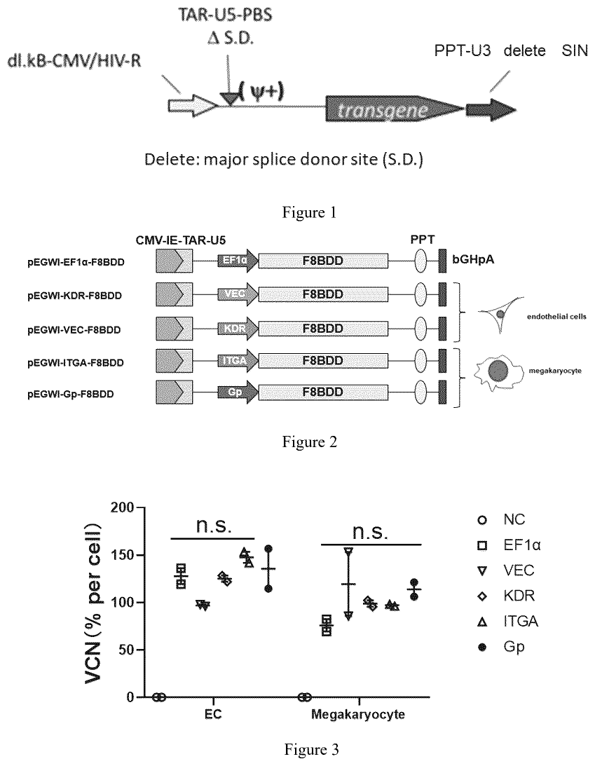

A Wasabi gene sequence (expressing a fluorescent protein), a B-domain-deleted F8 gene (F8-BDD) sequence (SEQ ID NO: 5), and nucleic acid sequences of tissue-specific promoters EF1α (SEQ ID NO: 6), VEC (SEQ ID NO: 1), KDR (SEQ ID NO: 2), ITGA (SEQ ID NO: 3) and Gp (SEQ ID NO: 4) were synthesized through whole-genome synthesis. Each of the above promoters and F8-BDD were co-ligated to the lentiviral vector pEGWI through a restriction enzyme site. The obtained products were identified by sequencing and double-enzyme digestions, referring to the original recommendation of New England Biolabs (NEB) for an optimal reaction condition. A BamHI cloning site (ggatccacc)-AUG was used at a 5′-end, and a SpeI cloning site (actagt) was used at a 3′-end. Correctly-ligated lentiviral vectors pEGWI-EF1a-F8-BDD, pEGWI-VEC-F8-BDD, pEGWI-KDR-F8-BDD, pEGWI-ITGA-F8-BDD or pEGWI-Gp-F8-BDD were obtained, in which the F8-BDD gene is driven under promoters including EF1a, VEC, KDR, ITGA or Gp, respectively. Specific ligation positions and compositions of the lentiviral vectors are shown in FIG. 2. Each promoter and the Wasabi gene were co-inserted into pEGWI to obtain lentiviral vectors pEGWI-EF1a-Wasabi, pEGWI-VEC-Wasabi, pEGWI-KDR-Wasabi, pEGWI-ITGA-Wasabi or pEGWI-Gp-Wasabi for use as controls in subsequent experiments.

SEQ ID NO: 6:
ataatggccataatcacgaattggccgcagatctcgaccaattctcatgtttgacagcttatcatcgataagctttggagctaagccagcaat
ggtagagggaagattctgcacgtcccttccaggcggcctccccgtcaccaccccccccaacccgccccgaccggagctgagagtaattcata
caaaaggactcgcccctgccttggggaatcccagggaccgtcgttaaactcccactaacgtagaacccagagatcgctgcgttcccgccccct
cacccgcccgctctcgtcatcactgaggtggagaagagcatgcgtgaggctccggtgcccgtcagtgggcagagcgcacatcgcccacagtc
cccgagaagttggggggaggggtcggcaattgaaccggtgcctagagaaagtggcgcggggtaaactgggaaagtgatgtcgtgtactggc
tccgcctttttcccgagggtgggggagaaccgtatataagtgcagtagtcgccgtgaacgttctttttcgcaacgggtttgccgccagaacaca
ggtaagtgccgtgtgtggttcccgcgggcctggcctctttacgggttatggcccttgcgtgccttgaattacttccacgcccctggctgcagta
cgtgattcttgatcccgagcttcgggttggaagtggggggagagttcgaggccttgcgcttaaggagccccttcgcctcgtgcttgagttgagg
cctggcctgggcgctggggccgccgcgtgcgaatctggtggcaccttcgcgcctgtctcgctgctttcgataagtctctagccatttaaaattt
ttgatgacctgctgcgacgctttttttctggcaagatagtcttgtaaatgcgggccaagatctgcacactggtatttcggtttttggggccgcg
ggcggcgacggggcccgtgcgtcccagcgcacatgttcggcgaggcggggcctgcgagcgcggccaccgagaatcggacgggggtagtctcaag
ctggccggcctgctctggtgcctggcctcgcgccgccgtgtatcgccccgccctgggcggcaaggctggcccggtcggcaccagttgcgtgagc
ggaaagatggccgcttcccggccctgctgcagggagctcaaaatggaggacgcggcgctcgggagagcgggcgggtgagtcacccacacaaa
ggaaaagggcctttccgtcctcagccgtcgcttcatgtgactccacggagtaccgggcgccgtccaggcacctcgattagttctcgagcttttg
gagtacgtcgtctttaggttggggggaggggttttatgcgatggagtttccccacactgagtgggtggagactgaagttaggccagcttggcac
ttgatgtaattctccttggaatttgccctttttgagtttggatcttggttcattctcaagcctcagacagtggttcaaagtttttttcttccat
ttcaggtgtcgtgaaaactctagagcggccgcggaggccgaattccgtcga.

### Example 2

In the present example, the lentiviral vectors constructed in Example 1 were further packaged, purified and concentrated to obtain recombinant lentiviruses. For an experimental method, see “[1] Chang L-J, Urlacher V, Iwakuma T, et al. Efficacy and safety analyses of a recombinant human immunodeficiency virus type 1 derived vector system [J]. Gene Therapy, 1999, 6 (5): 715-728” and “[2] Chang L-J, Zaiss A K. Chang, L-J and Zaiss, AK. Lentiviral vectors. Preparation and use. Methods Mol Med 69:303-318 [J]. Methods in Molecular Medicine, 2002, 69:303-318”.

For specific steps, reference may be made to the above literature. The specific steps are briefly described below.

(1) The lentiviral vectors constructed in Example 1 and packaging helper plasmids pNHP and pHEF-VSV-G were co-transduced into a mammalian cell HEK293T and cultured for 48 h, and the supernatant was collected.

(2) The collected lentiviruses were purified and concentrated to obtain the recombinant lentiviruses which were named LV-EF1a-F8-BDD, LV-VEC-F8-BDD, LV-KDR-F8-BDD, LV-ITGA-F8-BDD, LV-Gp-F8-BDD, LV-EF1a-Wasabi, LV-VEC-Wasabi, LV-KDR-Wasabi, LV-ITGA-Wasabi and LV-Gp-Wasabi, respectively.

(3) VCNs of the lentiviruses were detected, and the detection results are shown in FIG. 3. The lentiviruses LV-EF1a-F8-BDD, LV-VEC-F8-BDD, LV-KDR-F8-BDD, LV-ITGA-F8-BDD and LV-Gp-F8-BDD had basically similar VCNs at same multiplicity of infection (MOI).

### Example 3

In the present example, the recombinant lentiviruses containing different promoters and the Wasabi gene prepared in Example 2 were tested in vitro. The specificity of the promoters in different cells was detected by detecting the fluorescent protein amount expressed by the Wasabi gene.

Five lentiviruses (LV-EF1a-Wasabi, LV-VEC-Wasabi, LV-KDR-Wasabi, LV-ITGA-Wasabi and LV-Gp-Wasabi) carrying a normal Wasabi gene prepared in Example 2 were separately transduced into two cell lines: endothelial cells (ECs) and megakaryocytes. The transduction method of the lentiviruses is described below.

Dulbecco's modified eagle's medium (DMEM) containing 10% fetal bovine serum and a 1% penicillin-streptomycin solution was added to a six-well plate (Corning Incorporated, USA). 3×10⁴ ECs or 1×10⁵ megakaryocytes were inoculated in each well, cultured at 37° C. under 5% CO2 for 18 h, transduced with the lentiviruses at an MOI of 200, supplemented with polybrene (8 μg/mL, Sigma-Aldrich) to a final volume of 600 μL and transduced for 24 h. Then, the medium was replaced with a fresh medium every day. When the confluence of the cells reached 90%, the cells were moved to a T75 cm² culture flask (Corning Incorporated, USA).

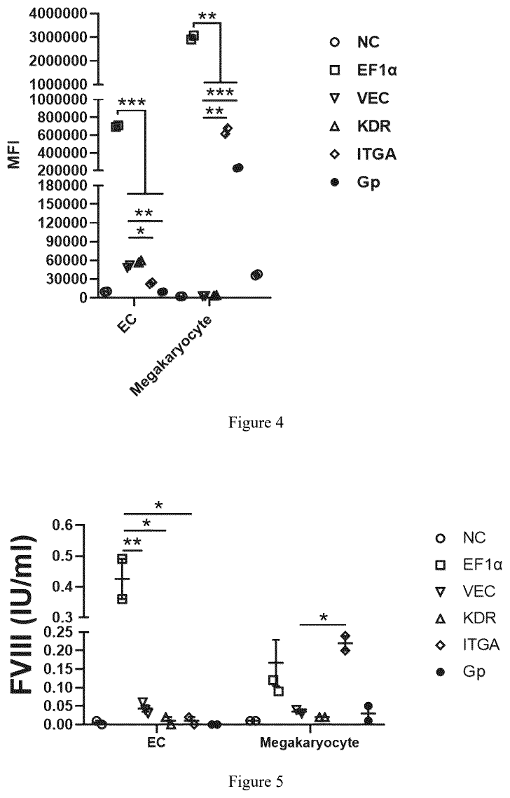

The amount of expressed fluorescent protein was detected to determine the expression of the Wasabi gene in the cells. The results are shown in FIG. 4. ECs transduced with LV-EF1α-Wasabi and megakaryocytes transduced with LV-EF1α-Wasabi both had high fluorescence intensities, indicating that the EF1α promoter efficiently promotes the expression of the Wasabi gene in both two types of cells and does not have tissue specificity. ECs transduced with LV-VEC-Wasabi and LV-KDR-Wasabi, respectively, had a higher fluorescence intensity than ECs transduced with LV-ITGA-Wasabi and LV-Gp-Wasabi, respectively. Megakaryocytes transduced with LV-VEC-Wasabi and LV-KDR-Wasabi, respectively, had a lower fluorescence intensity than megakaryocytes transduced with LV-ITGA-Wasabi and LV-Gp-Wasabi, respectively. To conclude, VEC and KDR promoters have EC specificity, and ITGA and Gp promoters have megakaryocyte specificity.

### Example 4

In the present example, the recombinant lentiviruses carrying the F8-BDD gene prepared in Example 2 were tested in vitro.

Five lentiviruses (LV-EF1α-F8-BDD, LV-VEC-F8-BDD, LV-KDR-F8-BDD, LV-ITGA-F8-BDD and LV-Gp-F8-BDD) carrying the F8-BDD gene prepared in Example 2 were separately transduced into two cell lines: endothelial cells (EA-hy926) and megakaryocyte (DAMI). The transduction method of the lentiviruses was the same as that in Example 3.

The supernatant secreted by transduced EA-hy926 and DAMI cells was collected and concentrated. At the same time, the intracellular extract was collected. The expression amount of the protein was detected through ELISA. Cells transduced with no lentiviruses were used as a negative control (NC). The results are shown in FIG. 5. The universal EF1α promoter efficiently promoted the expression of F8 in both two cells. In megakaryocytes, the ITGA promoter efficiently promoted the expression of F8. In ECs, the VEC promoter had a higher ability to promote the expression of F8 than other tissue-specific promoters, but F8 was only expressed at an extremely low level (10-fold lower) compared with the EF1α promoter.

A method for assessing a coagulation function is based on a substrate luminescence assay method, which is a method for assaying activity by using a human F8 chromogenic assay kit (Hyphen BioMed, France). The substrate luminescence assay method is as follows: plasma to be tested and a blank control group were diluted 40 times with a Tris-BSA buffer (R4+), 50 μL of each system was added to a microplate, a 50 μL X factor (R1), a 50 μL mixture (R2) of activated IX factors and a 50 μL SXa-11 substrate (R3) were added to the system, the system was incubated at 37° C. for 5 min, 50 μL of 20% acetic acid was added to stop the reaction, and an absorbance value was read at an absorbance of 405 nm.

The collected supernatant of EA-hy926 and DAMI transduced with the viruses was taken out at −80° C., thawed on ice, each supernatant was mixed with plasma of an F8-deficient individual, the plasma of the F8-deficient individual alone was used as an NC, plasma of a healthy volunteer was used as a positive control (PC), and the substrate luminescence method was used for detection.

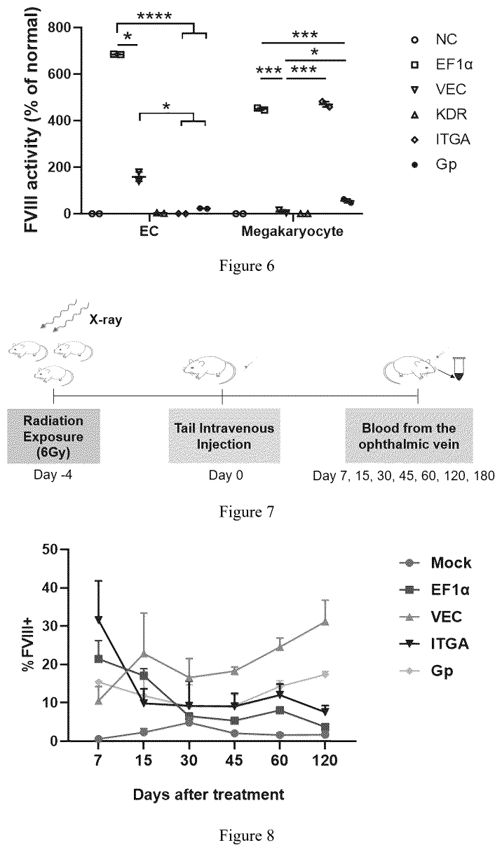

FIG. 6 illustrates detection results of human F8 through the substrate luminescence assay method. In the supernatant of EA-hy926 cells in which the expression of F8 was promoted by EF1α and the supernatant of EA-hy926 cells in which the expression of F8 was promoted by VEC, the activity of human F8 within a therapeutic range was detected and was about 6 times and 2.5 times higher than a normal level, respectively, while no activity of human F8 was detected in the supernatant of cells containing other promoters. In the supernatant of DAMI cells in which the expression of F8 was promoted by EF1α and the supernatant of DAMI cells in which the expression of F8 was promoted by ITGA, the activity of human F8 was detected to be 5 times higher than the normal level.

To conclude, in the present application, the expression of the F8 gene can be successfully promoted by the tissue-specific promoters of the lentiviral vectors so that a normal human F8 protein is expressed in the cells, and the VEC and ITGA promoters have relatively good specificity and promote an expression of a human F8 protein with potential of high activity and the coagulation function.

### Example 5

The lentiviruses carrying F8-BDD prepared in Example 2 were separately and directly injected into HA mice via tail veins to perform a treatment experiment.

The schematic diagram of treatment process of HA mice is shown in FIG. 7. The HA mice used were C57BL/6 female mice (6 weeks old, purchased from Beijing Biosubstrate Technologies) with F8 gene knockout. All mice were placed in a pathogen-free environment and irradiated (600 cGy/mouse) using an X-ray radiation cabinet (Faxitron, Tucson, AZ, USA). Lentiviruses LV-EF1α-F8-BDD, LV-VEC-F8-BDD, LV-ITGA-F8-BDD and LV-Gp-F8-BDD were separately injected into the HA mice through direct intravenous injection for treating the disease, where the injection doses of the viruses were 1×10⁷ TU. Phosphate buffered saline (PBS) (200 μL for each mouse) was used as a control (Mock).

On Days 7, 15, 30, 45, 60 and 120 after the treatment, the expression of human F8 gene in the peripheral blood was detected through flow cytometry. The results are shown in FIG. 8. Human F8 was stably expressed in the blood of mice in the LV-VEC-F8-BDD treatment group (10%˜30% of the normal plasma level). Human F8 was stably expressed also in the blood of mice in the LV-Gp-F8-BDD treatment group, at about 15%. While, the expression of the human F8 in the blood of mice in LV-EF1α-F8-BDD and LV-ITGA-F8-BDD treatment groups was gradually decreased (decreased from 30% to 5%).

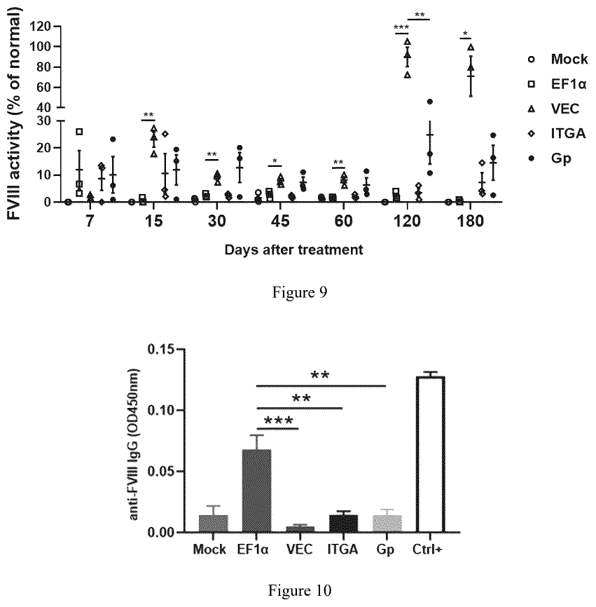

On Days 7, 15, 30, 45, 60 and 120 after the treatment, the blood was drawn from the mice, and plasma was separated from the blood. Untreated hemophilic mice (Mock) and wild-type mice (WT) were separately used as control, and the activity of F8 was measured through the substrate luminescence method. The results are shown in FIG. 9. The results were consistent with the results measured through the flow cytometry. In the LV-VEC-F8-BDD and LV-Gp-F8-BDD treatment groups, the activity of human F8 in mouse plasma stably increased, and reached a positive rate of 25% on Day 60, and further increased to 80% (the LV-VEC-F8-BDD treatment group) and 25% (the LV-Gp-F8-BDD treatment group) on Day 120. In the LV-EF1α-F8-BDD and LV-ITGA-F8-BDD treatment groups, the activity of human F8 in mouse plasma gradually decreased (less than 3%) after 30 days.

To conclude, both the detection results of the flow cytometry and the substrate luminescence method prove that tail vein injection of LV-Gp-F8-BDD or LV-VEC-F8-BDD allows F8 to be significantly improved and maintain at a stable level in the plasma of HA mice. That is, LV-Gp-F8-BDD or LV-VEC-F8-BDD can effectively treat HA in mice.

In addition, for an antibody response, the orbital peripheral blood of the above treated mice was collected and centrifuged at 3000 rpm for 15 min to obtain plasma. The plasma was diluted with a Tris-BSA buffer at a ratio of 1:200 and placed in a polyvinyl chloride (PVC) microplate. Peroxidase-conjugated goat anti-mouse total IgG was added. Then, a luminescent substrate 3,3′,5,5′-tetramethylbenzidine (TMB) was added for ELISA to evaluate the antibody response against coagulation factor VIII (F8). HA mice injected with an anti-coagulation factor VIII monoclonal antibody was used as a positive control (Ctrl+). The results are shown in FIG. 10. IgG antibody responses of the mice in the LV-VEC-F8-BDD, LV-Gp-F8-BDD and LV-ITGA-F8-BDD groups were all relatively low, and antibody responses of the mice in the LV-EF1α-F8-BDD group were the highest, indicating that gene therapy employing tissue-specific promoters VEC, Gp and ITGA of the present application can effectively reduce immune rejection.

To conclude, the tissue-specific promoter is creatively designed in the present application. The tissue-specific promoter can promote a specific expression of a coding gene in the EC or the megakaryocyte-platelet cell, effectively reduce an ectopic expression and can be applied to gene therapy in which the specific expression of the gene is required in the EC or the megakaryocyte-platelet cell, such as gene therapy for HA. The lentiviruses carrying the tissue-specific promoters and the F8-BDD gene are prepared in the present application. The HA mice are treated through the direct intravenous injection, effectively improving delivery efficiency of the F8-BDD gene and an amount of expressed F8-BDD gene in mice and correcting a hemorrhagic phenotype of the HA mice to a certain extent. The VEC promoter has the best therapeutic effect on promoting the expression of the F8-BDD gene and little antibody response, which is of great significance for ensuring effectiveness of gene therapy and lays a foundation for achieving faster relief from a symptom of HA and more comprehensive and durable gene therapy.

The applicant has stated that although the detailed method of the present application is described through the examples described above, the present application is not limited to the detailed method described above, which means that implementation of the present application does not necessarily depend on the detailed method described above. It should be apparent to those skilled in the art that any improvements made to the present application, equivalent replacements of raw materials of the product of the present application, additions of adjuvant ingredients to the product of the present application, and selections of specific manners, etc., all fall within the protection scope and the disclosure scope of the present application.

1. (canceled)

2. (canceled)

3. (canceled)

4. A recombinant expression vector, comprising a tissue-specific promoter and a coding gene;
wherein the tissue-specific promoter has the sequence as shown in SEQ ID NO: 1; and
the coding gene comprises a coding gene of a recombinant coagulation factor VIII, wherein the coding gene has the sequence as shown in SEQ ID NO: 5.

5. A recombinant lentivirus containing the recombinant expression vector according to claim 4.

6. A recombinant cell containing the recombinant expression vector according to claim 4.

7. (canceled)

8. A method for preparing the recombinant cell according to claim 6, comprising:
introducing the recombinant expression vector into a host cell to obtain the recombinant cell;
preferably, the introduction is carried out by a method which comprises any one of electrical transduction, a viral vector system, a non-viral vector system or direct gene injection; and
preferably, the host cell comprises a hematopoietic stem cell.

9. A pharmaceutical composition, comprising the recombinant expression vector according to claim 4; and
preferably, the pharmaceutical composition further comprises any one or a combination of at least two of a pharmaceutically acceptable carrier, excipient or diluent.

10. (canceled)

11. The recombinant expression vector according to claim 4, wherein the recombinant expression vector comprises a viral vector or a plasmid vector.

12. The recombinant expression vector according to claim 11, wherein the viral vector comprises a lentiviral vector pEGWI.

13. A method for treating hemophilia A, comprising administering the recombinant expression vector according to claim 4 to a patient in need thereof.


SEQUENCE LISTING (6 sequences)

SEQ ID NO: 1 - DNA, 1218 bp, synthetic construct
  source 1..1218: mol_type = other DNA
gcggccgctccaaaaatatccttccatcacactccccatcttgtgctctgatttactaaacggccctgggccctctctttctcagggtctctgcttgcccagctatataataaaacaagtttgggacttcccaaccattcacccatggaaaaacagaagcaactcttcaaaggacagattcccaggatctgccctgggagattccaaatcagttgatctggggtgagcccagtcctctgtagtttttagaagctcctcctatgtctctcctggtcagcagaatcttggcccctcccttccccccagcctcttggttcttctgggctctgatccagcctcagcgtcactgtcttccacgcccctctttgattctcgtttatgtcaaaagccttgtgaggatgaggctgtgattatccccattttacagatgaggaaactgtggctccaggatgacacaactggccagaggtcacatcagaagcagagctgggtcacttgactccacccaatatccctaaatgcaaacatcccctacagaccgaggctggcaccttagagctggagtccatgcccgctctgaccaggagaagccaacctggtcctccagagccaagagcttctgtccctttcccatctcctgaagcctccctgtcacctttaaagtccattcccacaaagacatcatgggatcaccacagaaaatcaagctctggggctaggctgaccccagctagatttttggctcttttataccccagctgggtggacaagcaccttaaacccgctgagcctcagcttcccgggctataaaatgggggtgatgacacctgcctgtagcattccaaggagggttaaatgtgatgctgcagccaagggtccccacagccaggctctttgcaggtgctgggttcagagtcccagagctgaggccgggagtaggggttcaagtggggtgccccaggcagggtccagtgccagccctctgtggagacagccatccggggccgaggcagccgcccaccgcagggcctgcctatctgcagccagcccagccctcacaaaggaacaataacaggaaaccatcccagggggaagtgggccagggccagctggaaaacctgaaggggaggcagccaggcctccctcgccagcggggtgtggctcccctccaaagacggtcggctgacaggctccacagagctccactcccggggatcc

SEQ ID NO: 2 - DNA, 1098 bp, synthetic construct
  source 1..1098: mol_type = other DNA
gcggccgcctccttcccctgggcctaaggatatcttggctggaagctctgctctgaaaaggggcatggccaaactttcactagggctcttcgttggggagcacgatggacaaaagccttcttggggctaggcaggtcacttcaaacttggagccgccaaatattttgggaaatagcgggaatgctggcgaactgggcaagtgcgttttctgattaagagcaaccagattcagctttttaaactacaattatactggccaaacaaaatacccttatacaaaaaccaaaactactggcaggagtcgctgccagcttgcgacccggcatacttggctgagtatccgcttctcccttgtggctccaaactgctgcagattctcggccacttcagacgcgcgcgatggcgaagagggtcctgcactttgacgcgcctggtgagggagcggtgctcttcgcagcgctcctggtgatgctccccaaatttcggggaccggcaagcgattaaatcttggagttgctcagcgcccgttaccgagtactttttatttacaccagaaacaaagttgttgctctgggatgttctctcctgggcgacttggggcccagcgcagtccagttgtgtggggaaatggggagatgtaaatgggcttggggagctggagatccccgccgggtacccgggtgaggggcggggctggccgcacgggagagcccctcctccgccccggccccgccccgcatggccccgcctccgcgctctagagtttcggctccagctcccaccctgcactgagtcccgggaccccgggagagcggtcagtgtgtggtcgctgcgtttcctctgcctgcgccgggcatcacttgcgcgccgcagaaagtccgtctggcagcctggatatcctctcctaccggcacccgcagacgcccctgcagccgccggtcggcgcccgggctccctagccctgtgcgctcaactgtcctgcgctgcggggtgccgcgagttccacctccgcgcctccttctctagacaggcgctgggagaaagaaccggctcccgagttctgggcatttcgcccggctcgaggtgccccggggatcc

SEQ ID NO: 3 - DNA, 963 bp, synthetic construct
  source 1..963: mol_type = other DNA
gcggccgctgtgaacggaccaagagtaaacagtgtgctcaatgctgtgcctacgtgtgttagcccacgcggccagcctgaggagtcagggaaggctcccctaggcaaagcccccaaccagaatcaagtcttaatggttaaagagctccatcacccaaaaaggattgagggcctaccttcaactgaacagctaatgcataatctcagaaactgtgagtcaaaattccctggaataactccactttatccccaatctccttgccacctagaccaaggtccattcaccaccctgtccccagcactgactgcactgctgtggccacactaaagcttggctcaagacggaggaggagtgaggaagctgctgcaccaatatggctggttgaggccgcccaaggtcctagaaggaggaagtgggtaaatgccatatccaaaaagatacagaagcctcaggttttatcgggggcagcagcttccttctccttccccgacctgtggccaagtcacaaagcaccacagctgtacagccagatgggggaagggaggagattagaactgtaggctagagtagacaagtatggaccagttcacaatcacgctatcccaagcagaaagtgatggtggcttggactagcacggtggtagtagagatggggtaaagattcaagagacatcattgataggcagaaccaataggacatggtaataaactattctcaggaaaggggaggagtcatggctttcagccatgagcatccaccctctgggtggcctcacccacttcctggcaattctagccaccatgagtccaggggctatagccctttgctctgcccgttgctcagcaagttacttggggttccagtttgataagaaaagacttcctgtggaggaatctgaagggaaggaggaggagctggcccattcctgcctgggaggttgtggaagaaggacccggggatcc

SEQ ID NO: 4 - DNA, 340 bp, synthetic construct
  source 1..340: mol_type = other DNA
gcggccgctctgggattacaggcatgagccacgcgcccggccctggagaggtttttaaaagatggcagaaggctgtttggaggagtccacccccatctcccctgtgtaaaaggaaagcggaagagagaaccacaaagagggcctgggggaaagccgtggagtgaggcgataagggcttgtgtccaggggattcccggtcactggaatccctatcaggcctgcatttcctcctcacccccatccccttccttgccactggcttagtcctccatggggctagaagagagaaggacggagtcgagtggcaccctagaagacgctctgtgccttcggaggatcc

SEQ ID NO: 5 - DNA, 4374 bp, synthetic construct
  source 1..4374: mol_type = other DNA
atgcagatcgaactgagcacctgcttcttcctgtgtctcctgagattctgctttagtgctaccagacggtattacctgggagccgtcgagctgagttgggattacatgcagtccgacctcggagaactgcctgtggatgcacgctttccaccaagagtgcctaagtcattcccattcaacacctcagtcgtgtataagaagactctgttcgtcgagtttactgatcacctgttcaatatcgctaaacctagaccaccctggatgggactgctgggtcctacaatccaggcagaggtctatgacactgtggtgattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttttctcagaatcctcctgtcctcaaacgccatcaacgggagattacacggaccacactccaaagcgatcaggaggagatcgactatgacgataccatatctgtggaaatgaagaaagaggacttcgacatctacgacgaagatgagaaccaaagtccaagatccttccagaagaagactaggcactacttcatcgctgccgtggaacgcctctgggattacggaatgtccagttctccacatgtcctcaggaatagggcacagtctggctctgttccacagtttaagaaagttgtctttcaggagttcacagatggctcattcactcaaccactgtatagaggcgaactgaatgagcacctgggactgctgggtccctacatcagagccgaagtggaggataacattatggtcacctttcggaaccaagcctccaggccatacagtttctacagttctctgatctcatacgaggaagatcagaggcaaggagcagaaccaaggaagaacttcgtgaaaccaaacgagacaaagacctatttctggaaagttcagcatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 6 - DNA, 1545 bp, synthetic construct
  source 1..1545: mol_type = other DNA
ataatggccataatcacgaattggccgcagatctcgaccaattctcatgtttgacagcttatcatcgataagctttggagctaagccagcaatggtagagggaagattctgcacgtcccttccaggcggcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactctagagcggccgcggaggccgaattccgtcga